(19) United States
(12) Patent Application Publication
(10) Pub. No.: US 2013/0172973 A1
(43) Pub. Date: Jul. 4, 2013

(54) NEURAL STIMULATION DEVICE WITH INSULATING SHEATH

(71) Applicants: Bruce A. Tockman, Scandia, MN (US); Brian D. Soltis, St. Paul, MN (US); Lili Liu, Maple Grove, MN (US); Eric F. Hammill, Ham Lake, MN (US)

(72) Inventors: Bruce A. Tockman, Scandia, MN (US); Brian D. Soltis, St. Paul, MN (US); Lili Liu, Maple Grove, MN (US); Eric F. Hammill, Ham Lake, MN (US)

(21) Appl. No.: 13/728,366

(22) Filed: Dec. 27, 2012

Related U.S. Application Data
(60) Provisional application No. 61/582,938, filed on Jan. 4, 2012.

(51) Int. Cl. A61N 1/05

(52) U.S. Cl. CPC A61N 1/0556
USPC 607/118

(57) ABSTRACT

Described is a stimulation electrode assembly for stimulation of a vagus nerve within a carotid sheath. The stimulation electrode assembly comprises an insulating sheath and at least one energy delivery element. The insulating sheath includes a flexible sheet of electrically insulative material configured to assume a generally tubular shape to at least partially surround the carotid sheath when implanted, and having a first side and a second side, wherein the first side is positionable toward the carotid sheath when implanted. The at least one energy delivery element is located on the first side of the insulating sheath and is configured to deliver energy to the vagus nerve when implanted.

### CROSS-REFERENCE TO RELATED APPLICATION

This application claims priority to Provisional Application No. 61/582,938, filed Jan. 4, 2012, which is herein incorporated by reference in its entirety.

### TECHNICAL FIELD

The present disclosure relates to a device for neurostimulation of a selected nerve that avoids undesired stimulation of proximate nerves, tissues or muscles adjacent to the target nerve. More particularly, the present disclosure relates to a device, system and corresponding method for neurostimulation of a vagus nerve by surrounding a carotid sheath with an insulating sheath and providing an electrical stimulation device within the insulating sheath.

### BACKGROUND

The use of nerve stimulation for treating and controlling a variety of medical, psychiatric, and neurological disorders has seen significant growth over the last several decades, including for treatment of heart conditions, epilepsy, obesity, and breathing disorders, among others. For example, modulation of the autonomic balance with neural stimulation has been shown to be possible and have positive clinical benefits, such as protecting the myocardium from further remodeling and predisposition to fatal arrhythmias following a myocardial infarction (MI).

### SUMMARY

In Example 1, a stimulation electrode assembly for stimulation of a vagus nerve within a carotid sheath. The stimulation electrode assembly comprises an insulating sheath and at least one energy delivery element. The insulating sheath includes a flexible sheet of electrically insulative material configured to assume a generally tubular shape to at least partially surround the carotid sheath when implanted, and having a first side and a second side, wherein the first side is positionable toward the carotid sheath when implanted. The at least one energy delivery element is located on the first side of the insulating sheath and is configured to deliver energy to the vagus nerve when implanted.

In Example 2, the stimulation electrode assembly according to Example 1, wherein the at least one energy delivery element comprises a portion of a lead including at least one pair of electrodes.

In Example 3, the stimulation electrode assembly according to Example 1 or 2, wherein the at least one energy delivery element comprises a plurality of electrodes that are embedded within the insulating sheath.

In Example 4, the stimulation electrode assembly according to Examples 1-3, wherein the insulating sheath comprises a tube having a longitudinal axis and an opening along the longitudinal axis.

In Example 5, the stimulation electrode assembly according to Examples 1-4, the insulating sheath further comprising a first edge and an opposite second edge, wherein the first and second edges are configured to be joined in order to maintain the generally tubular shape of the insulating sheath.

In Example 6, the stimulation electrode assembly according to Example 5, wherein the first and second edges of the insulating sheath are configured to be joined by sutures.

In Example 7, the stimulation electrode assembly according to Examples 1-6, wherein the flexible sheet comprises a material having shape memory such that the flexible sheet has a predetermined tubular shape configured to at least partially surround the carotid sheath when implanted.

In Example 8, a stimulation electrode assembly for stimulation of a vagus nerve within a carotid sheath. The stimulation electrode assembly comprises an insulating sheath and at least one energy delivery element. The insulating sheath includes a flexible sheet of electrically insulative material configured to assume a generally tubular shape to at least partially surround the vagus nerve when implanted, and having a first side and a second side, wherein the first side is positionable toward the vagus nerve when implanted. The at least one energy delivery element is located on the first side of the insulating sheath that is configured to deliver energy to the vagus nerve.

In Example 9, the stimulation electrode assembly according to Example 8, wherein the at least one energy delivery element comprises a portion of a lead including at least one pair of electrodes.

In Example 10, the stimulation electrode assembly according to Example 8 or 9, wherein the at least one energy delivery element comprises a plurality of electrodes that are embedded within the insulating sheath.

In Example 11, the stimulation electrode assembly according to Examples 8-10, wherein the insulating sheath comprises a tube having a longitudinal axis and an opening along the longitudinal axis.

In Example 12, the stimulation electrode assembly according to Examples 8-11, the insulating sheath further comprising a first edge and an opposite second edge, wherein the first and second edges are configured to be joined in order to maintain the generally tubular shape of the insulating sheath.

In Example 13, the stimulation electrode assembly according to Example 12, wherein the first and second edges of the insulating sheath are configured to be joined by sutures.

In Example 14, the stimulation electrode assembly according to Examples 8-13, wherein the flexible sheet comprises a material having shape memory such that the flexible sheet has a predetermined tubular shape configured to at least partially surround the vagus nerve when implanted.

In Example 15, a method for stimulating a vagus nerve within a carotid sheath. The method comprising: positioning a stimulation electrode assembly at least partially around the carotid sheath or at least partially around the vagus nerve itself, the stimulation electrode assembly including: an insulating sheath comprising a flexible sheet of electrically insulative material configured to assume a generally tubular shape to at least partially surround the carotid sheath or at least partially surround the vagus nerve itself, and having a first side and a second side, wherein the first side is positioned toward the carotid sheath or the vagus nerve; and at least one energy delivery element located on the first side of the insulating sheath configured to deliver energy to the vagus nerve; and, energizing the at least one energy delivery element to direct energy toward the vagus nerve, wherein the insulating sheath inhibits conduction of energy beyond the carotid sheath during energization of the at least one energy delivery element.

In Example 16, the method according to Example 15, wherein the step of positioning the stimulation electrode assembly around the carotid sheath comprises accessing the carotid sheath via a cut-down procedure.

In Example 17, the method according to Example 15 or 16, wherein the insulating sheath further comprises a first edge and a second edge that may be joined in order to maintain the generally tubular shape of the insulating sheath, and further comprising the step of joining the first edge and the second edge of the insulating sheath together after the insulating sheath is positioned around the carotid sheath or the vagus nerve.

In Example 18, the method according to Examples 15-17, further comprising the step of suturing the insulating sheath around the carotid sheath or the vagus nerve.

In Example 19, the method according to Examples 15-18, further comprising the step of selectively energizing the at least one energy delivery element.

In Example 20, the method according to Examples 15-19, wherein the device is connected to a pulse generator that controls the at least one energy delivery element.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a system for stimulating a region of a vagus nerve located within the carotid sheath according to an embodiment.

FIG. 2A is a schematic view of a device for stimulating a region of a vagus nerve in an unassembled or open configuration.

FIG. 2B is a schematic view of the device of FIG. 2A in an assembled or closed configuration.

FIG. 3A is a schematic view of an embodiment of a device for stimulating a region of a vagus nerve.

FIG. 3B is a schematic view of an embodiment of a device for stimulating a region of a vagus nerve.

FIG. 3C is a schematic view of an embodiment of a device for stimulating a region of a vagus nerve.

FIG. 4 is a schematic view of an embodiment of a device for stimulating a region of a vagus nerve.

FIG. 5 is a schematic view of an embodiment of a device for stimulating a region of a vagus nerve.

FIG. 6 is a schematic view of a system for stimulating a region of a vagus nerve located within the carotid sheath according to an embodiment.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

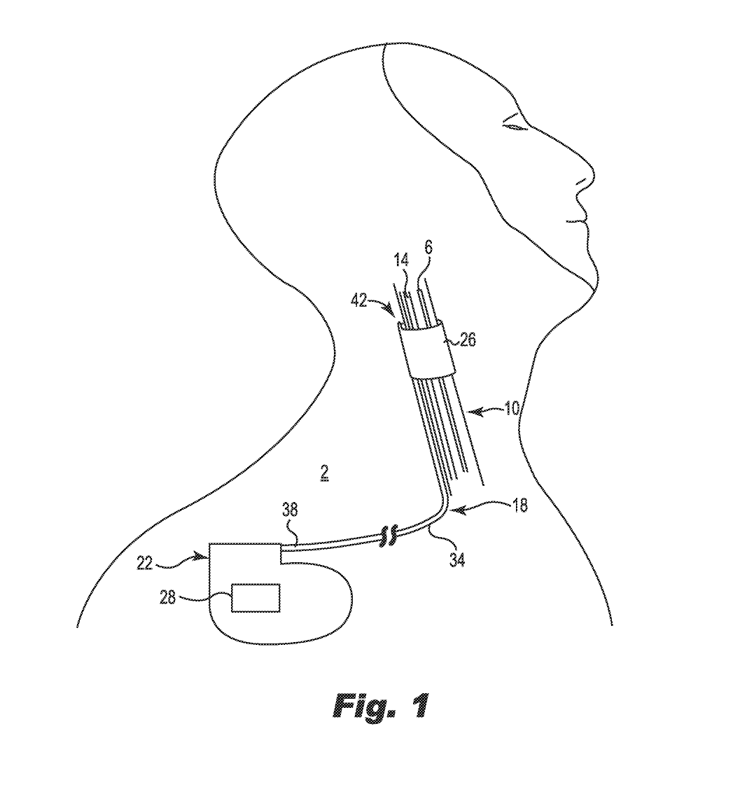

FIG. 1 is a schematic illustration showing a system 2 for stimulating a region of a patient's vagus nerve 6 located within a carotid sheath 10, which consists of multiple layers of fascia wrapping the common carotid artery 14, the internal jugular vein (IJV) (not shown), and the vagus nerve 6. As shown, the system 2 includes a lead 18, an implantable pulse generator 22 and an insulating sheath 26. In various embodiments, and as explained in greater detail herein, the lead 18 and the insulating sheath 26 cooperate to form a stimulation electrode assembly. In the illustrated embodiment, the lead 18 is coupled to the pulse generator 22, which includes a power source or battery 28. Additionally, the insulating sheath 26 is disposed about and surrounds the carotid sheath 10.

In the various embodiments, the system 2 can be used to selectively stimulate the vagus nerve 6, e.g., for treating cardiac disease. As such, in various embodiments, the lead 18 or the insulating sheath 26 include electrodes (not shown in FIG. 1) that are electrically and operatively coupled to electronics and the power supply 28 of the pulse generator 22 to deliver electrical stimuli to the vagus nerve 6 when implanted. Additionally, as shown in FIG. 1, the insulating sheath 26 surrounds the carotid sheath 10 when implanted and operates to inhibit undesired stimulation of nerves or muscle tissue outside the carotid sheath by preventing electrical stimuli emitted from the aforementioned electrodes from being directed to such nerves or tissues. Thus, the insulating sheath 26 operates to confine the stimuli to the contents of the carotid sheath 10. The system 2 thus facilitates providing optimal stimulation results by directing the stimuli towards the desired target—e.g., the vagus nerve 6—while minimizing conduction of energy to unintended anatomical structures.

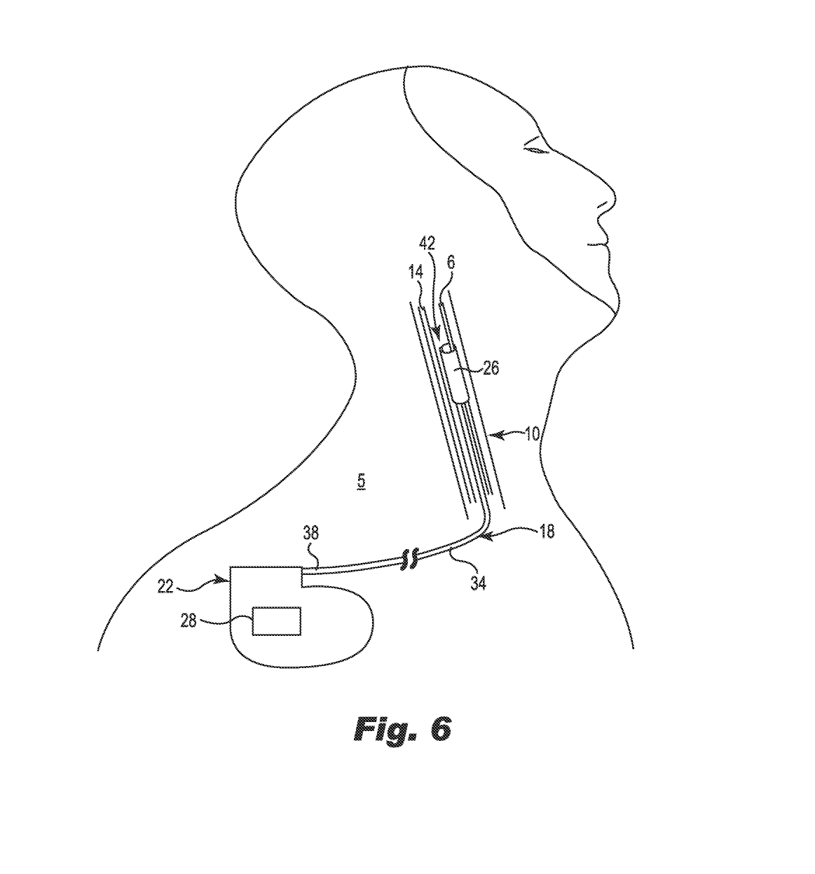

FIG. 6 shows an alternative system 5 to system 2 shown in FIG. 1. In system 5, the insulating sheath 26 is placed within the carotid sheath 10 and surrounds the vagus nerve 6. Alternatively, however, the insulating sheath 26 could also or alternatively surround at least one other component within the carotid sheath 10, such as the IJV (not shown) and/or the carotid artery 14.

As will be further explained herein, in various embodiments, the lead 18 and the insulating sheath 26 can be provided as separate elements that are coupled together in situ during the implantation process. Alternatively, in various embodiments, the lead 18 and the insulating sheath 26 can be a unitary element, with the lead 18 housing electrical conductors that are electrically connected to electrodes on the insulating sheath 26. All embodiments of the lead 18 and the insulating sheath 26 described herein may be incorporated into either system 2 (FIG. 1) or system 5 (FIG. 6), for example.

As shown in FIG. 1, the lead 18 includes an elongated, insulative lead body 34 extending from a proximal end 38 to a distal end 42. The lead 18 is coupled to the pulse generator 22 via a connector (not shown) located at the proximal end 38 of lead body 34. In various embodiments, the lead body 34 is generally flexible to allow for patient movement. In some embodiments, the lead body 34 can include one or more guide lumens to receive a guide member such as a guidewire or stylet in order to stiffen the lead body 34 for surgical implantation.

According to various embodiments, the lead 18 can include a plurality of conductors including individual wires, coils, or cables extending within the lead body 34 from the proximal end 38 in a direction towards the distal end 42 of the lead body 34. The conductors can be insulated with an insulator such as silicone, polyurethane, ethylene tetrafluoroethylene, or another biocompatible, insulative polymer. In one exemplary embodiment, the conductors have a co-radial design. In this embodiment, each individual conductor is separately insulated and then wound together in parallel to form a single coil. In another exemplary embodiment, the conductors have a co-axial, non-co-radial configuration. In various embodiments, the individual conductors may be single or multi-filar coil conductors. In still other embodiments, one or more of the conductors is a stranded cable conductor each routed through one of the aforementioned lumens in the lead body 34. In short, the various embodiments are not limited to any particular conductor configuration within the lead 18.

In various embodiments, the insulating sheath 26 can have a number of suitable configurations that are able to effectively surround the carotid sheath 10, or components within the carotid sheath 10, such as the vagus nerve 6, and substantially prevent undesired stimulation of nerves or other anatomical structures outside the carotid sheath 10. As shown in FIG. 1, after being implanted in the patient and assembled, the insulating sheath 26 has a generally tubular shape that can surround or encircle a portion or the entire circumference of the carotid sheath 10 (or the vagus nerve 6, as shown in FIG. 6). In the illustrated embodiment, the insulating sheath 26 is configured to be wrapped fully around the carotid sheath 10, but in alternative embodiments the insulating sheath 26 can be configured to wrap around only a portion of the circumference of the carotid sheath 10 (or a portion of the vagus nerve 6).

The insulating sheath 26 can be formed from a sheet of flexible, insulative material. Suitable polymers that may be used for the insulating sheath 26 include, for example, silicone, polyurethane, polysiloxane urethane, ethylene tetrafluoroethylene (ETFE), polytetrafluoroethylene (PTFE), expanded PTFE (ePTFE), and expanded ultra-high-molecular-weight polyethylene (eUHMWPE), although others are also contemplated. Alternatively, the insulating sheath 26 could comprise a flexible sheet of material with an outer surface(s) or coating(s) of insulating material. Examples of such insulating materials include, but are not limited to those listed above that may be used for the insulating sheath 26. Depending upon the configuration of the assembled insulating sheath and the method of assembling the sheath, the desired flexibility of the material may vary.

As assembled, the insulating sheath 26 can be generally tubular-shaped or cylindrically-shaped. The tubular or cylindrical shape of the insulating sheath 26 can also be tapered, such that when assembled, the diameter of the insulating sheath 26 at one end is greater than the diameter at the opposite end. In various embodiments, the material forming the insulating sheath 26 may have a uniform or non-uniform cross-sectional thickness. In various embodiments, the insulating sheath 26 can be configured to exhibit a shape memory so as to be biased toward its final assembled (e.g., tubular) shape when implanted.

Although in the illustrated embodiment of system 2, the insulating sheath 26 surrounds the exterior of the carotid sheath 10, in other embodiments the insulating sheath 26 and the stimulation electrodes can be deployed within the carotid sheath 10, such as in system 5 (see FIG. 6). The insulating sheath 26 can surround certain components or contents within the carotid sheath 10, such as the vagus nerve 6 by itself (as shown in FIG. 6), the vagus nerve 6 and the carotid artery 14, the vagus nerve 6 and the IJV, or the vagus nerve 6, carotid artery 14 and the IJV, for examples.

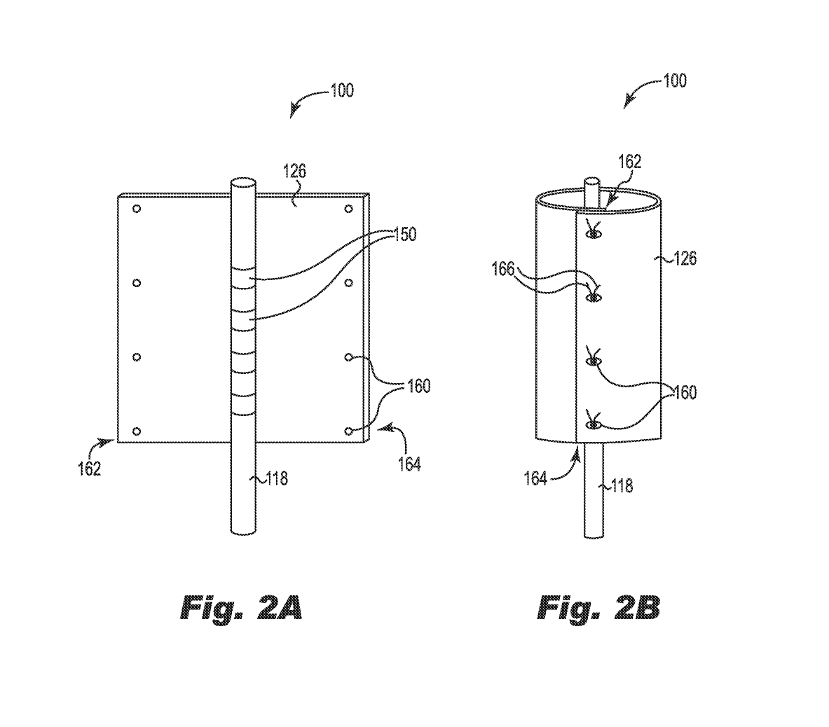

FIGS. 2A and 2B are schematic illustrations showing a portion of stimulation electrode assembly 100 including an implantable lead 118 and an insulating sheath 126 in open (or unassembled) and closed (or assembled) configurations, respectively, according to one embodiment. The stimulation electrode assembly 100 can be used in system 2 or 5 described previously, i.e., in the same manner as the lead 18 and the insulating sheath 26 described in connection with FIG. 1.

In the illustrated embodiments, the insulating sheath 126 can be a flexible, polymer sheet, and the lead 118 can be bonded, attached or joined to the insulating sheath 126 such that when assembled, the lead 118 can be joined to an inner surface of the insulating sheath 26. The lead 118 can include a plurality of electrodes 150 that are used to stimulate the vagus nerve. In various embodiments, the electrodes 150 can be ring or partial ring electrodes. When partial-ring electrodes are utilized, the lead 118 is oriented such that the electrode 150 is oriented in a direction towards the region of the vagus nerve 6, or other target nerve structure, to be stimulated. In the various embodiments, the electrodes 150 and the portion of the lead 118 positioned within the insulating sheath 126 constitute an energy delivery element of the stimulation electrode assembly 100.

As shown, the insulating sheath 126 can also include a plurality of suture holes 160 along first 162 and second edges 164. The suture holes 160 are arranged such that joining the sutures holes from first and second edges 162, 164 together results in assembly of the insulating sheath 126. When assembled, the insulating sheath 126 forms a generally cylindrical or tubular shape that can surround the carotid sheath (see FIG. 1) or vagus nerve 6 (see FIG. 6).

To assemble the insulating sheath 126 around the carotid sheath 10 (see FIG. 1) or vagus nerve 6 (see FIG. 6), the first edge 162 of the insulating sheath 126, including suture holes 160, and the second edge 164, including complementary suture holes 160, are urged around the carotid sheath 10 (see FIG. 1) or vagus nerve 6 (see FIG. 6) and then joined together. The first and second edges 162, 164 are joined by lining up the holes 160 on the first and second edges 162, 164 and suturing them together using sutures 166 (FIG. 2B). Other alternative means for joining first edge 162 and second edge 164, are, however, also contemplated. For example, the first edge 162 and the second edge 164 of the insulating sheath 126 may instead be joined or attached using an adhesive, a hook and loop fastening mechanism, or other comparable joining techniques or features. In various embodiments, sutures can be pre-attached to the insulating sheath 126 at the first edge 162 and the second edge 164. The sutures could be used by a surgeon in order to pull the insulating sheath 126 around the carotid sheath 10 or vagus nerve 6, with the sutures being tied together to secure the first and second edges 162, 164 together.

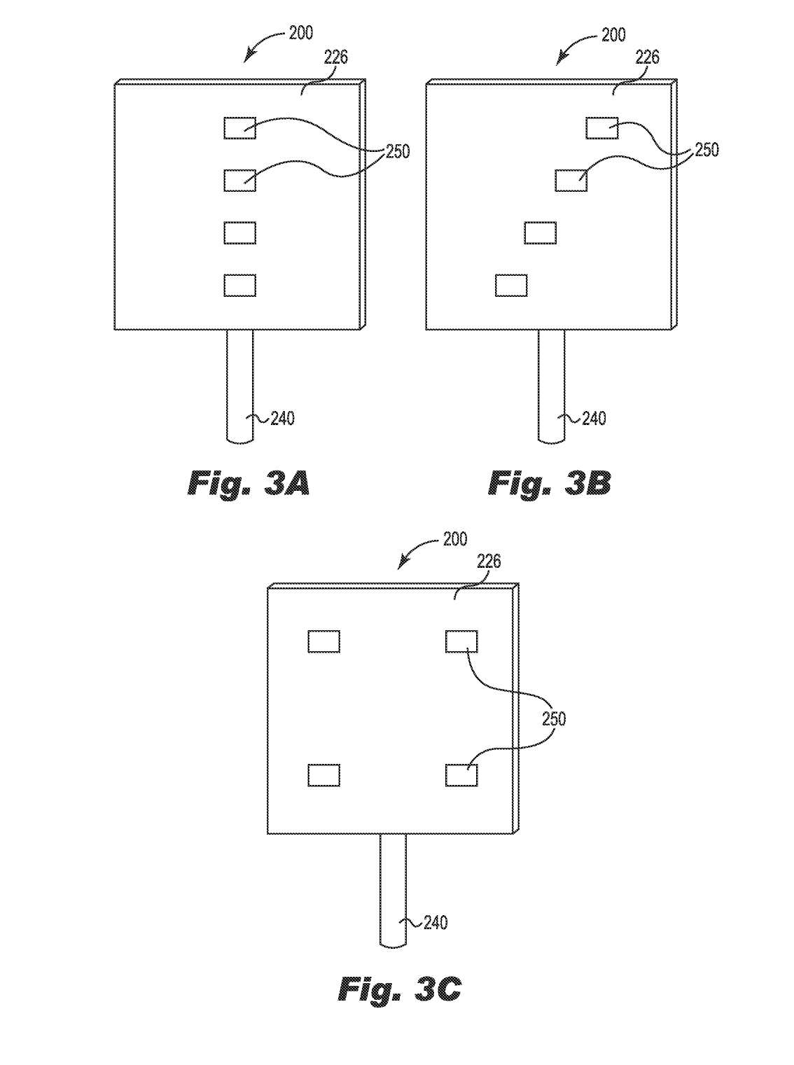

FIGS. 3A, 3B and 3C are schematic illustrations of portions of alternative embodiments of a stimulation electrode assembly 200 in an unassembled/undeployed state. As shown, the stimulation electrode assembly 200 includes insulating sheath 226, a lead 240 and a plurality of electrodes 250. The stimulation electrode assembly 200 can be used in systems 2 or 5 described previously, i.e., in the same manner as the lead 18 and the insulating sheath 26 described in connection with FIG. 1. Except as otherwise described in connection with FIGS. 3A-3C, the insulating sheath 226 can be substantially similar to the insulating sheath 126 described previously with regard to the embodiment shown in FIGS. 2A and 2B. In the illustrated embodiment, the electrodes 250 are located proximate an inner surface of the insulating sheath 226. In various embodiments, the electrodes 250 can be disposed on and bonded to the inner surface of the insulating sheath 226. In other embodiments, the electrodes 250 can be partially or fully embedded within the insulating sheath 226 with only an active surface of the electrodes 250 exposed (i.e., not surrounded by the insulative material of the insulating sheath 226). The inner surface of the insulating sheath 226 is configured as the surface that will be oriented toward the carotid sheath 10 (see FIG. 1) or vagus nerve 6 (see FIG. 6) when the device 200 is assembled around the carotid sheath 10 or vagus nerve 6, respectively.

As shown, the lead 240 can be coupled to the insulating sheath 226. As previously discussed, the stimulation electrode assembly 200 can be used in the system 2 illustrated in FIG. 1 or system 5 illustrated in FIG. 6, such that the stimulation electrode assembly 200 can be used in the same manner as the lead 18 and the insulating sheath 26 described in connection with FIG. 1. As such, in various embodiments, the lead 240 can be coupled to the pulse generator 22 (see FIGS. 1 and 6) to supply electrical energy to the electrodes 250. The lead 240 operates to electrically couple the electrodes 250 to the pulse generator 22 (see FIGS. 1 and 6), i.e., via electrical conductors disposed within the lead 240 and extending into the insulating sheath 226 to the respective electrodes 250. In various embodiments, the electrodes 250 constitute energy delivery elements of the stimulation electrode assembly 200.

In various embodiments, the lead 240 can be integrally formed with the insulating sheath 226. For example, in various embodiments, the insulating sheath 226 can be integrally formed with the outer insulating material (e.g., silicone rubber) of the lead 240. In other embodiments, the insulating sheath 226, including the electrodes 250 and corresponding electrical conductors (not shown) connected thereto can be formed separately from the lead 240, and the insulating sheath 226 and the lead 240, and the respective electrical conductors, can be connected as a separate manufacturing step.

In the embodiment of FIG. 3A, the electrodes 250 extend generally linearly along the insulating sheath 226 at or near the center of the insulating sheath 226. FIG. 3B shows an alternative arrangement in which the electrodes 250 are disposed in a staggered configuration or distribution. FIG. 3C shows another alternative arrangement of the electrodes 250, with the electrodes 250 being arranged in a square pattern. The square pattern includes two columns of two electrodes 250 each (although other numbers of electrodes 250 are also contemplated), with the columns being parallel to one another. Once the insulating sheath 226 is wrapped around the carotid sheath 10 (as in FIG. 1), the two columns can be located about 180° apart surrounding the carotid sheath 10. If insulating sheath 226 is instead wrapped directly around vagus nerve 6 (as in FIG. 6), the two columns can be located about 180° apart surrounding the vagus nerve 6. As a result of either such a configuration, electrical energy can be delivered between two electrodes 250 in the same column, which delivers energy longitudinally along the vagus nerve 6, or the energy can be delivered between two electrodes 250 in different columns, which delivers energy across the assembled insulating sheath 226 and the vagus nerve 6.

The particular electrode configurations shown in FIGS. 3A-3C are exemplary only, and in various other embodiments, still other electrode orientations and positions can be employed. Although FIGS. 3A-3C illustrate embodiments utilizing four electrodes 250, in various embodiments, more or fewer electrodes can be utilized.

In the illustrated embodiments, the stimulation electrode assembly 200 includes a separate insulating sheath 226 and a lead 240 coupled to the pulse generator 22 (see FIG. 1). However, it is also contemplated that the electrodes 250 can be coupled directly to the pulse generator without requiring an intervening lead 240. For example, the pulse generator 22 of FIG. 1 could be attached directly to an outer surface of the insulating sheath 226 and the electrodes 250 could be electrically coupled to the pulse generator 22 via a connection header on the pulse generator 22.

In the various embodiments of the stimulation electrode assemblies 100, 200, the associated electrodes can be formed of any conventional implantable lead electrode materials—e.g., platinum, platinum iridium, titanium, etc. The various embodiments are not limited to any particular electrode material.

In various embodiments, system 2 or 5, utilizing the stimulation electrode assembly 100 or 200, can be configured in a uni-polar arrangement, or can be configured in a multi-polar arrangement such as bipolar, tri-polar, quad-polar, or in an electrode array. Conductors extending within the respective leads 118, 240 can be adapted to be connected to each individual electrode in a one-to-one manner allowing each electrode to be individually addressable. Additionally, the pulse generator 22 can be programmed such that each respective electrode assumes a positive (+) or negative (−) polarity to create a particular stimulation field when current, for example, is applied thereto. During implantation, the physician may select the combination of electrodes that will cause an electric field to reach to the target neurological structure.

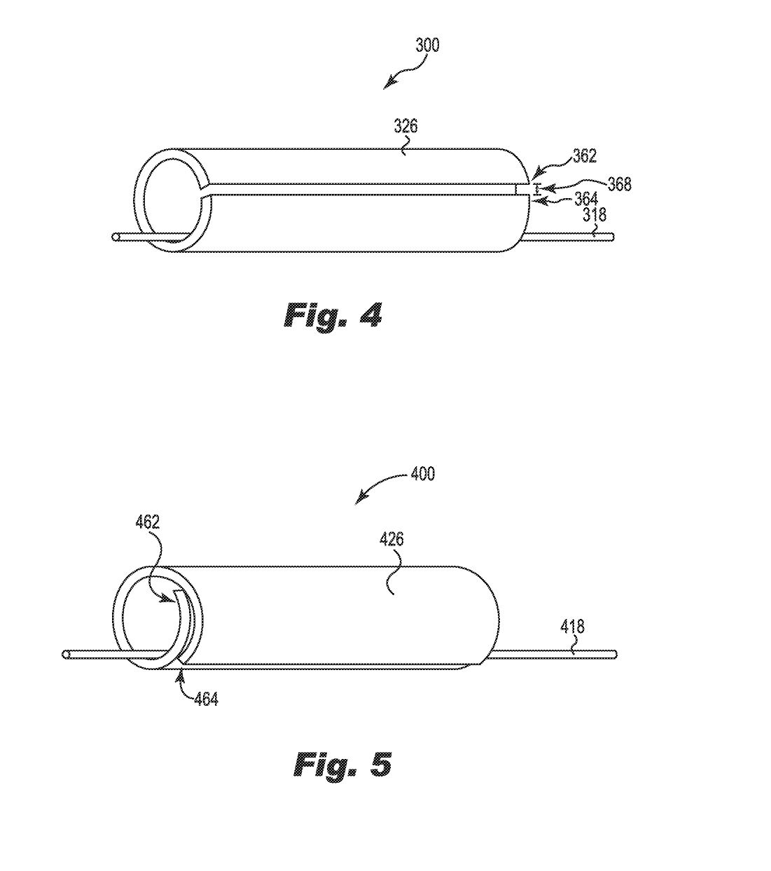

FIG. 4 is a schematic illustration of a stimulation electrode assembly 300 according to another embodiment. In the embodiment of FIG. 4, the stimulation electrode assembly 300 includes a lead 318 and an insulating sheath 326 having a predetermined tubular or cylindrical shape. The stimulation electrode assembly 300 can be used in system 2 or 5 described previously, i.e., in the same manner as the lead 18 and the insulating sheath 26 described in connection with FIG. 1. The material used for the insulating sheath 326 can have shape memory or may be formed in such a way as to return to a predetermined tubular shape. As shown, the insulating sheath 326 includes a first edge 362 and a second edge 364 that are adjacent one another in the predetermined tubular shape. In the illustrated embodiment, an opening or gap 368 is present between the first and second edges 362, 364 in order for the insulating sheath 326 to be opened to be fit around a carotid sheath and then allowed to close to surround the carotid sheath 10 (see FIG. 1) or vagus nerve 6 (see FIG. 6). The opening 368 can be closed upon assembly of the insulating sheath 326 around the carotid sheath.

In various embodiments, the lead 318 can be substantially similar or identical to the leads 18, 118 described previously. Alternatively, in various embodiments, the stimulation electrode assembly 300 may include a plurality of electrodes on the insulating sheath 326 and a lead such as the lead 240 of FIG. 3A, 3B or 3C in lieu of the lead 318 extending into the insulating sheath 326.

FIG. 5 is a schematic illustration of a stimulation electrode assembly 400 according to another embodiment. As shown in FIG. 5, the stimulation electrode assembly 400 includes a lead 418 and an insulating sheath 426 similar to the insulating sheath 326 in the embodiment of FIG. 4. The stimulation electrode assembly 400 can be used in system 2 described previously, i.e., in the same manner as the lead 18 and the insulating sheath 26 described in connection with FIG. 1, or in system 5 (FIG. 6), for example. The insulating sheath 426 can be formed of a material having shape memory or may be formed in such a way that the insulating sheath 426 returns to a predetermined tubular shape (i.e., is pre-coiled). As shown, the insulating sheath 426 includes a first edge 462 and a second edge 464. As further shown, when deployed, the insulating sheath 426 overlaps a portion of itself. In various embodiments, the insulating sheath 426 can be unrolled to fit around the carotid sheath or vagus nerve, or other carotid sheath components, during implantation, and then is allowed to spiral around itself to close.

In various embodiments, the insulating sheath 426 is configured to retain its shape so as to engage and remain wrapped around the carotid sheath or vagus nerve or other carotid sheath component without requiring additional retention means. However, in various embodiments, the stimulation electrode assembly 400 can include additional retaining means, e.g., sutures, an adhesive, a hook and loop fastening mechanism, or other comparable joining features (not shown), for preventing the insulating sheath 426 from unrolling. In various embodiments, the lead 418 can be substantially similar or identical to the leads 18, 118 described previously. Alternatively, in various embodiments, the stimulation electrode assembly 400 may include a plurality of electrodes on the insulating sheath 426 and a lead such as the lead 240 of FIG. 3A or 3B, for example, in lieu of the lead 418.

According to an exemplary implantation method, the various stimulation electrode assemblies described herein (e.g., any of the stimulation electrode assemblies 100, 200, 300, 400) can be implanted though a small incision formed in the skin of the neck. In order to position the device around the carotid sheath, implantation requires cutting down close to the carotid sheath. After cutting down close to the carotid sheath, a space is then cleared 360° around the carotid sheath in order to allow the stimulation electrode assembly to be assembled around the carotid sheath. The insulating sheath is then wrapped fully or partially around the carotid sheath. In some embodiments, implantation of the stimulation electrode assembly does not require opening or entering the carotid sheath, which reduces the complexity of the implantation surgical procedure. In addition, if removal of the stimulation electrode assembly is necessary, it may be easier to extract than if the stimulation electrode assembly is implanted within the carotid sheath.

The various stimulation electrode assemblies may, however, alternatively be positioned within the carotid sheath, and surrounding one or more of the vagus nerve, the carotid artery and the IJV. FIG. 6 illustrates one example of the stimulation electrode assembly being implanted to surround the vagus nerve. The stimulation electrode assembly can be implanted between tissue or layers of fascia of the carotid sheath. A trocar can be used, for example, to separate fibers or tissue in order to implant the insulating sheath within the carotid sheath.

The stimulation electrode assembly may be sutured to or within the carotid sheath to hold the stimulation electrode assembly in place. Alternatively, an anchor or suture sleeve may be included on a lead or a conductor lumen proximal to the insulating sheath in order to prevent longitudinal movement of the stimulation electrode assembly. Other methods of attachment of the stimulation electrode assembly within the patient's body are also contemplated.

In the case of system 2 or 5, lead 18 may be tunneled to a pulse generator 22 (see FIGS. 1 and 6). The lead 18 is inserted into the pulse generator 22 and secured. The pulse generator 22 is typically implanted subcutaneously or submuscularly within an implantation location or pocket in the patient's pectoral region on the same side of the body as the incision in the neck needed to access the carotid sheath 10. The lead 18 is tunneled from the area of the carotid sheath 10 over the clavicle and down into the pulse generator 22 pocket. The lead 18 extends from the pulse generator 22 to the insulating sheath 26 surrounding the carotid sheath 10 and is also located subcutaneously or submuscularly. Any excess lead 18 length can be coiled up in the subcutaneous pocket near the pulse generator 22.

The configuration of the stimulation electrode assembly allows for it to be repositioned as necessary until an optimal position for stimulation resulting in a desired physiological effect is identified. For example, the patient can be monitored to determine if the desired physiological response to vagus nerve stimulation is achieved. In addition, physiological effects of undesired stimulation, e.g., laryngeal muscle vibration from activation of the recurrent laryngeal nerve (RLN) (branch of the vagus nerve), can be monitored. If less than optimal physiological responses are detected, the stimulation electrode assembly can be repositioned.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A stimulation electrode assembly for stimulation of a vagus nerve within a carotid sheath, the stimulation electrode assembly comprising:
an insulating sheath including a flexible sheet of electrically insulative material configured to assume a generally tubular shape to at least partially surround the carotid sheath when implanted, and having a first side and a second side, wherein the first side is positionable toward the carotid sheath when implanted; and
at least one energy delivery element located on the first side of the insulating sheath that is configured to deliver energy to the vagus nerve.

2. The stimulation electrode assembly of claim 1, wherein the at least one energy delivery element comprises a portion of a lead including at least one pair of electrodes.

3. The stimulation electrode assembly of claim 1, wherein the at least one energy delivery element comprises a plurality of electrodes that are embedded within the insulating sheath.

4. The stimulation electrode assembly of claim 1, wherein the insulating sheath comprises a tube having a longitudinal axis and an opening along the longitudinal axis.

5. The stimulation electrode assembly of claim 1, the insulating sheath further comprising a first edge and an opposite second edge, wherein the first and second edges are configured to be joined in order to maintain the generally tubular shape of the insulating sheath.

6. The stimulation electrode assembly of claim 5, wherein the first and second edges of the insulating sheath are configured to be joined by sutures.

7. The stimulation electrode assembly of claim 1, wherein the flexible sheet comprises a material having shape memory such that the flexible sheet has a predetermined tubular shape configured to at least partially surround the carotid sheath when implanted.

8. A stimulation electrode assembly for stimulation of a vagus nerve within a carotid sheath, the stimulation electrode assembly comprising:
an insulating sheath including a flexible sheet of electrically insulative material configured to assume a generally tubular shape to at least partially surround the vagus nerve when implanted, and having a first side and a second side, wherein the first side is positionable toward the vagus nerve when implanted; and
at least one energy delivery element located on the first side of the insulating sheath that is configured to deliver energy to the vagus nerve.

9. The stimulation electrode assembly of claim 8, wherein the at least one energy delivery element comprises a portion of a lead including at least one pair of electrodes.

10. The stimulation electrode assembly of claim 8, wherein the at least one energy delivery element comprises a plurality of electrodes that are embedded within the insulating sheath.

11. The stimulation electrode assembly of claim 8, wherein the insulating sheath comprises a tube having a longitudinal axis and an opening along the longitudinal axis.

12. The stimulation electrode assembly of claim 8, the insulating sheath further comprising a first edge and an opposite second edge, wherein the first and second edges are configured to be joined in order to maintain the generally tubular shape of the insulating sheath.

13. The stimulation electrode assembly of claim 12, wherein the first and second edges of the insulating sheath are configured to be joined by sutures.

14. The stimulation electrode assembly of claim 8, wherein the flexible sheet comprises a material having shape memory such that the flexible sheet has a predetermined tubular shape configured to at least partially surround the vagus nerve when implanted.

15. A method for stimulating a vagus nerve within a carotid sheath, comprising:
positioning a stimulation electrode assembly at least partially around the carotid sheath or at least partially around the vagus nerve itself, the stimulation electrode assembly including:
an insulating sheath comprising a flexible sheet of electrically insulative material configured to assume a generally tubular shape to at least partially surround the carotid sheath or at least partially surround the vagus nerve itself, and having a first side and a second side, wherein the first side is positioned toward the carotid sheath or the vagus nerve; and
at least one energy delivery element located on the first side of the insulating sheath configured to deliver energy to the vagus nerve; and,
energizing the at least one energy delivery element to direct energy toward the vagus nerve, wherein the insulating sheath inhibits conduction of energy beyond the carotid sheath during energization of the at least one energy delivery element.

16. The method of claim 15, wherein the step of positioning the stimulation electrode assembly around the carotid sheath comprises accessing the carotid sheath via a cut-down procedure.

17. The method of claim 15, wherein the insulating sheath further comprises a first edge and a second edge that may be joined in order to maintain the generally tubular shape of the insulating sheath, and further comprising the step of joining the first edge and the second edge of the insulating sheath together after the insulating sheath is positioned around the carotid sheath or the vagus nerve.

18. The method of claim 15, further comprising the step of suturing the insulating sheath around the carotid sheath or the vagus nerve.

19. The method of claim 15, further comprising the step of selectively energizing the at least one energy delivery element.

20. The method of claim 15, wherein the device is connected to a pulse generator that controls the at least one energy delivery element.
